(19) United States
(12) Patent Application Publication
(10) Pub. No.: US 2024/0112777 A1
(43) Pub. Date: Apr. 4, 2024

(54) HEALTH SUPPORT APPARATUS, HEALTH SUPPORT SYSTEM, AND HEALTH SUPPORT METHOD

(71) Applicant: MICIN, Inc., Tokyo (JP)

(72) Inventors: Mie KUNIO, Tokyo (JP); Kentaro HANAKI, Tokyo (JP); Tadaatsu HANADATE, Tokyo (JP)

(21) Appl. No.: 18/263,344

(22) PCT Filed: Jan. 28, 2022
(86) PCT No.: PCT/JP2022/003389

§ 371 Date: Jul. 28, 2023

(30) Foreign Application Priority Data
Jan. 29, 2021 (JP) 2021-013966

(51) Int. Cl. G16H 20/00; G16H 10/60

(52) U.S. Cl. CPC G16H 20/00; G16H 10/60

(57) ABSTRACT

A health support apparatus for supporting symptomatic alleviation or maintenance and improvement of the physical condition of the user includes: a life information reception unit configured to receive life information including information on activity that the user performs in daily life; an analysis unit configured to identify actionable behavior that the user can perform in the activity; and a presentation unit configured to present the actionable behavior to the user or the medical professional.

### FIELD

The present disclosure relates to a health support apparatus, a health support system, and a health support method.

### BACKGROUND

A technique for supporting physicians in diagnosis is known.

### CITATION LIST

### Patent Literature

Patent Literature 1: Japanese Patent Application Laid-open No. 2020-17137

### SUMMARY

### Technical Problem

The technique described above is for estimating the name of a highly possible disease from symptoms and findings and recommending effective tests and inspections to physicians. Unfortunately, even if the disease is actually identified and an appropriate treatment is guided to patients, there is the problem that due to the diversity of values, lifestyle rhythms, and genetic diversity, patients do not practice the guidance or the effectiveness is limited even if the patients practice the instruction.

The present disclosure is made in view of the above problems, and an object thereof is to analyze actionable behavior (a method of intervention) for a patient or a person who is subject to maintenance and improvement of a physical condition (hereinafter referred to as a user) to allow the user or a medical professional to utilize resultant information to aim for symptomatic alleviation or maintenance and improvement of a physical condition of the user.

### Solution to Problem

According to the present disclosure, a health support apparatus for supporting symptomatic alleviation or maintenance and improvement of a physical condition of a user is provided. The health support apparatus includes a life information reception unit configured to receive life information including information on activity that the user performs in daily life, an analysis unit configured to identify the activity that is continuously performed as actionable behavior, and a presentation unit configured to present the actionable behavior to the user or a medical professional as a candidate of a method of intervention for the user.

### Advantageous Effects of Invention

According to the present disclosure, actionable behavior (a method of intervention) can be analyzed for a user, and the user or a medical professional can utilize resultant information to aim for symptomatic alleviation or maintenance and improvement of a physical condition of the user.

### BRIEF DESCRIPTION OF DRAWINGS

FIG. 1 is a diagram illustrating an overall configuration example of a health support system according to the present embodiment.

FIG. 2 is a diagram illustrating a hardware configuration example of a computer for implementing a server apparatus 1 according to the embodiment.

FIG. 3 is a diagram illustrating a software configuration example of the server apparatus 1 according to the embodiment.

FIG. 4 is a diagram illustrating a configuration example of information stored in a user information storage unit 131 according to the embodiment.

FIG. 5 is a diagram illustrating a configuration example of information stored in a vital information storage unit 132 according to the embodiment.

FIG. 6 is a diagram illustrating a configuration example of information stored in a life information storage unit 133 according to the embodiment.

FIG. 7 is a diagram illustrating a configuration example of information stored in a guidance information storage unit 134 according to the embodiment.

FIG. 8 is a flowchart diagram according to a series of controls in the server apparatus 1 according to the embodiment.

### DESCRIPTION OF EMBODIMENTS

The contents of embodiments of the present invention will be listed and described. One embodiment of the present invention includes the following configurations.

### [Note 1]

A health support apparatus for supporting symptomatic alleviation or maintenance and improvement of a physical condition of a user, the health support apparatus including:
a life information reception unit configured to receive life information including information on activity that the user performs in daily life;
an analysis unit configured to identify actionable behavior that the user is able to perform in the activity; and
a presentation unit configured to present the actionable behavior to the user or a medical professional.

### [Note 2]

The health support apparatus according to note 1, in which
the life information includes information on emotion of the user when the activity has been performed,
the analysis unit increases priority of actionable behavior accompanied by non-negative emotion in the actionable behavior, and
the presentation unit presents the actionable behavior for which the analysis unit has increased priority in a manner distinguishable from the other actionable behavior.

### [Note 3]

The health support apparatus according to note 1 or 2, further including:
a vital information reception unit configured to acquire vital information of the user; and
a symptom determination unit configured to determine a change in a symptom of the user from the vital information, in which
the analysis unit identifies the actionable behavior performed before a time when the symptom determination unit determines that the symptom has improved, as symptom-related actionable behavior, and
the presentation unit presents the symptom-related actionable behavior in a manner distinguishable from the actionable behavior.

### [Note 4]

The health support apparatus according to any one of Notes 1 to 3, in which the analysis unit statistically estimates the actionable behavior.

### [Note 5]

The health support apparatus according to any one of Notes 1 to 4, further including:
a user information reception unit configured to receive user information on an attribute of the user; and
a model generation unit configured to estimate the actionable behavior of the user.

### [Note 6]

The health support apparatus according to note 5, in which
the model generation unit uses the actionable behavior as correct data and generates a prediction model in which input data is the user information and the life information and output is behavior that the user is able to perform, and
the presentation unit presents the behavior output by the prediction model.

### [Note 7]

The health support apparatus according to note 5, in which
the model generation unit uses the symptom-related actionable behavior as correct data and generates a prediction model in which input data is the user information and the life information and output is behavior that the user is able to perform, and
as correct data the presentation unit presents the behavior output by the prediction model.

### [Note 8]

A health support system for supporting symptomatic alleviation or maintenance and improvement of a physical condition of a user, the health support system including:
a life information reception function to receive life information including information on activity that the user performs in daily life;
an analysis function to identify actionable behavior that the user is able to perform in the activity; and
a presentation function to present the actionable behavior to the user or a medical professional.

### [Note 9]

A health support method for supporting symptomatic alleviation or maintenance and improvement of a physical condition of a user,
the health support method performed by a processor including:
a life information receiving step of receiving life information including information on activity that the user performs in daily life;
an analyzing step of identifying actionable behavior that the user is able to perform in the activity; and a presenting step of presenting the actionable behavior to the user or a medical professional.

A detailed description is given below on suitable embodiments of the present disclosure with reference to the accompanying drawings. In this specification and the drawings, redundant explanations of components having substantially identical functional configurations are omitted by applying the same symbols.

A server apparatus 1 of the present embodiment identifies actionable behavior for a user. The term actionable described here includes not only that the user can continuously perform, but also that the user can continuously stop performing, and further that the user can continue to change behavior. Further, it is also included that after the user has received guidance (intervention) by the medical professional, the activity that the user has followed in the guidance is determined.

==Overview==

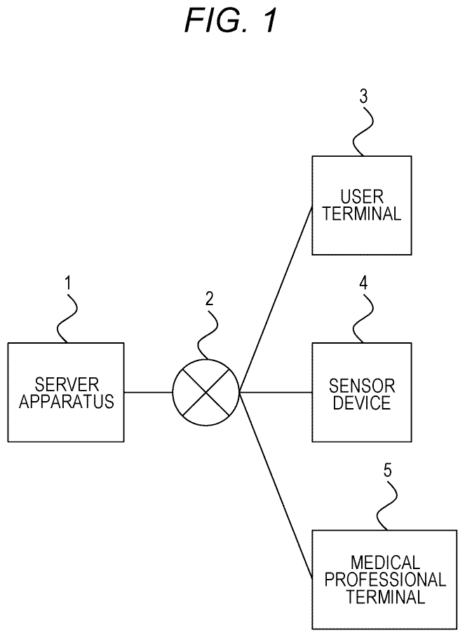

FIG. 1 is a diagram illustrating an overall configuration of the server apparatus 1 (an information processing apparatus). As illustrated in FIG. 1, the health support system includes the server apparatus 1, a user terminal 3, a sensor device 4, and a medical professional terminal 5. The server apparatus 1 is connected to the user terminal 3, the sensor device 4, and the medical professional terminal 5 via a network 2. Although only one user terminal 3, one sensor device 4, and one medical professional terminal 5 are illustrated, it is apparent that there may be more user terminals, sensor devices, and medical professional terminals. In addition, specific devices of the user terminal 3, the sensor device 4, and the medical professional terminal 5 are not limited to a mobile terminal and a personal computer and, for example, may be a smartphone, a tablet computer, a wearable terminal, or any other electronic devices.

==User Terminal 3==

The user terminal 3 is a computer that is operated by a patient or a person (a user) who is subject to symptomatic alleviation or maintenance and improvement of a physical condition. The user terminal 3 is, for example, a smartphone, a tablet computer, or a personal computer. The user can access the server apparatus 1, for example, through an application executed on the user terminal 3 or a Web browser.

==Sensor Device 4==

The sensor device 4 is a computer having a sensor for acquiring live data of the user. Acceptable sensors for the system of the present disclosure can vary widely, and may include both a sensor in continuity with and attached to the body of a patient and a sensor remote to the body of the patient. Possible sensors may include accelerometers, RFID sensing, resistive, capacitive, inductive, and magnetic sensors, reflective sensors, infrared sensors, video monitoring, pressure, and stress sensors, transcutaneous oxygen pressure sensors, transcutaneous CO₂ sensors, hydration sensors, pH sensors, ultrasound sensors, remote optical spectroscopy sensors, laser Doppler flow sensors, GPS, and the like. The user terminal 3, which is equipped with the sensor described above, may also serve as the sensor device 4.

==Medical Professional Terminal 5==

The medical professional terminal 5 is a computer that is operated by a medical professional with whom the user consults regarding health, the medical professional including a physician, a dentist, a pharmacist, a health nurse, a midwife, a nurse, an assistant nurse, a physiotherapist, an occupational therapist, an orthoptist, a speech therapist, a prosthetist, a radiology therapist, a clinical laboratory technologist, a clinical engineer, a licensed masseur, an acupuncturist, a judo therapist, an emergency life guard, and the like. The medical professional terminal 5 is, for example, a smartphone, a tablet computer, or a personal computer. The medical professional can access the server apparatus 1, for example, through an application executed on the medical professional terminal 5 or a Web browser.

Hereinafter, a configuration of the server apparatus 1 will be described.

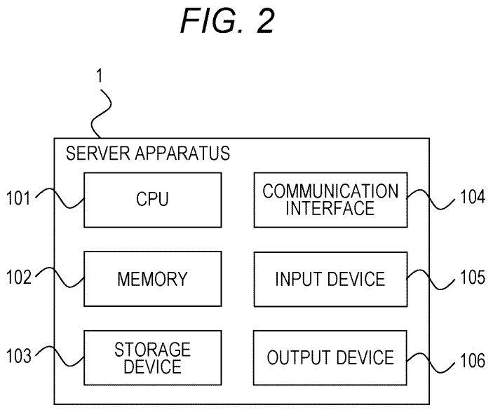

FIG. 2 is a diagram illustrating a hardware configuration example of the server apparatus 1 of the present embodiment. The server apparatus 1 includes a CPU 101, a memory 102, a storage device 103, a communication interface 104, an input device 105, and an output device 106. The CPU 101 is an arithmetic device that controls the operation of the entire server apparatus 1, controls the transmission and reception of data between each element, and performs information processing necessary for application execution and authentication processing. For example, the CPU 101 is processor such as a central processing unit (CPU), and performs various types of information processing by executing a program or the like stored in the storage device 103 and loaded into the memory 102. The memory 102 includes a main memory composed of a volatile storage device such as a dynamic random access memory (DRAM) and an auxiliary memory composed of a non-volatile storage device such as a flash memory or a hard disc drive (HDD). The memory 102 is used as a work area or the like of the CPU 101, and stores a basic input/output system (BIOS) executed when the server apparatus 1 is started, various setting information, and the like. The storage device 103 is, for example, a hard disk drive, a solid state drive, or a flash memory, for storing various data and programs. The communication interface 104 is an interface for connecting to the network 2, such as an adapter for connecting to Ethernet (registered trademark), a modem for connecting to a public telephone network, a wireless communication device for performing wireless communication, a universal serial bus (USB) connector for serial communication, or an RS232C connector. The input device 105 is a device for receiving data input through, for example, a keyboard or mouse, a touch panel, a button, or a microphone. The output device 106 includes, for example, a display, a printer, or a speaker, for outputting data.

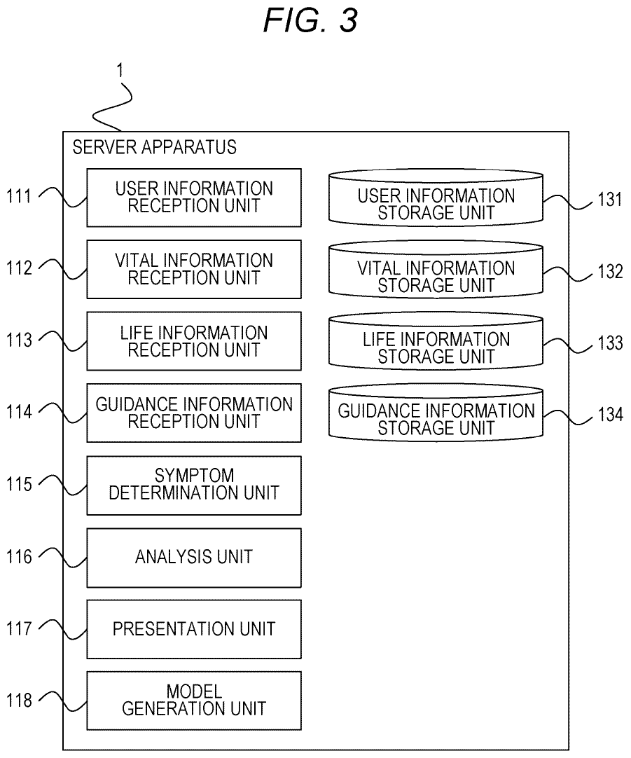

FIG. 3 is a block diagram illustrating a functional configuration of the server apparatus 1. As illustrated in FIG. 3, the server apparatus 1 includes functional units, that is, a user information reception unit 111, a vital information reception unit 112, a life information reception unit 113, a guidance information reception unit 114, a symptom determination unit 115, an analysis unit 116, a presentation unit 117, and a model generation unit 118, and storage units, that is, a user information storage unit 131, a vital information storage unit 132, a life information storage unit 133, a guidance information storage unit 134, and an analysis data storage unit 135.

The functional units described above are implemented through reading-out of a program stored in the storage device 103 to the memory 102 and execution of the program by the CPU 101 included in the server apparatus 1. The storage units described above are implemented as part of storage areas provided by the memory 102 and the storage device 103 included in the server apparatus 1.

Here, in the present embodiment, a data configuration of the following storage units will be illustrated: the user information storage unit 131, the vital information storage unit 132, the life information storage unit 133, the guidance information storage unit 134, and the analysis data storage unit 135.

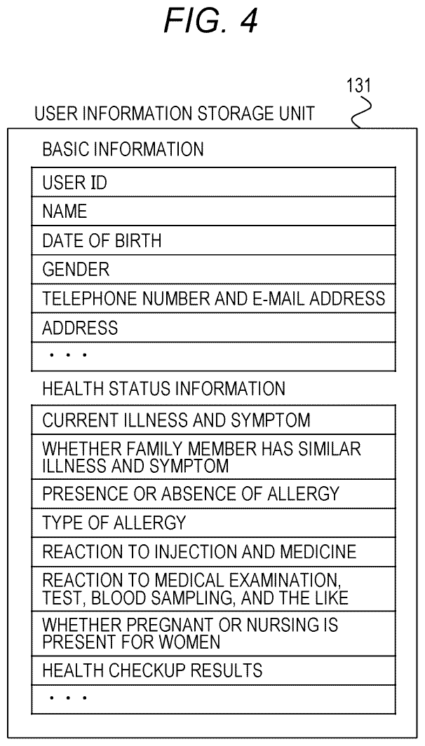

The user information storage unit 131 stores user information, an example of which is illustrated in FIG. 4, received by the user information reception unit 111. As illustrated in FIG. 4, the user information is information that illustrates attributes and health status of a user, and may be, for example, information that is written and input in a medical interview sheet at a medical examination in a hospital or the like. As an example, the user information broadly includes, but not limited to, basic information and health status information. The basic information is configured from information such as a user ID, a name, a date of birth, gender, a telephone number and an e-mail address, an address, an emergency contact person, and relationship to the emergency contact person, as an example. The health status information is configured from information such as current illnesses and symptoms, whether family members have similar illnesses and symptoms, the presence or absence of allergies, the types of allergies, reactions to injections and medications, reactions to medical examinations, tests, blood sampling, and the like, whether pregnant or nursing is present for women, and information acquired by general health checkup.

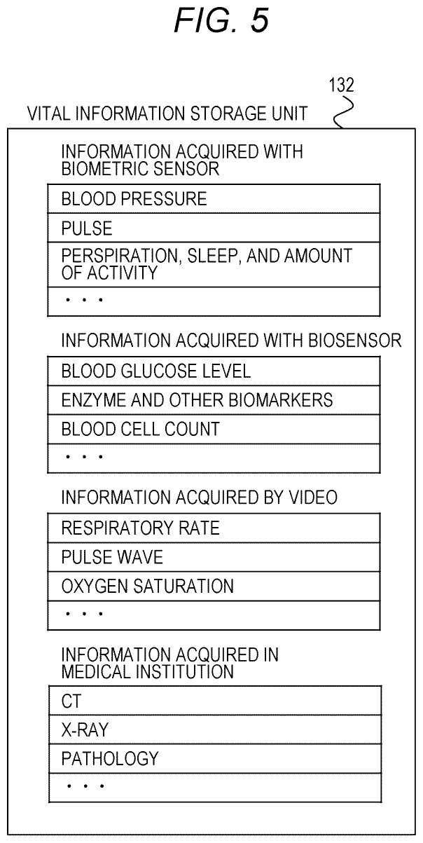

The vital information storage unit 132 stores vital information, an example of which is illustrated in FIG. 5. received by the vital information reception unit 112, As illustrated in FIG. 5, the vital information is information that objectively illustrates the status of a user. As an example, the vital information broadly includes, but not limited to, information acquired with a biometric sensor, information acquired with a biosensor, information acquired by video, and information acquired in a medical institution.

The information acquired with a biometric sensor is configured from information such as blood pressure, pulse, perspiration, sleep, and the amount of activity, as an example. The information acquired with a biosensor is configured from information such as blood glucose levels, enzymes and other biomarkers, and blood cell counts, as an example. The information acquired by video is configured from information such as respiratory rate, pulse wave, and oxygen saturation, as an example. The information acquired in a medical institution is configured from information such as CT, X-ray, and pathological examination, as an example.

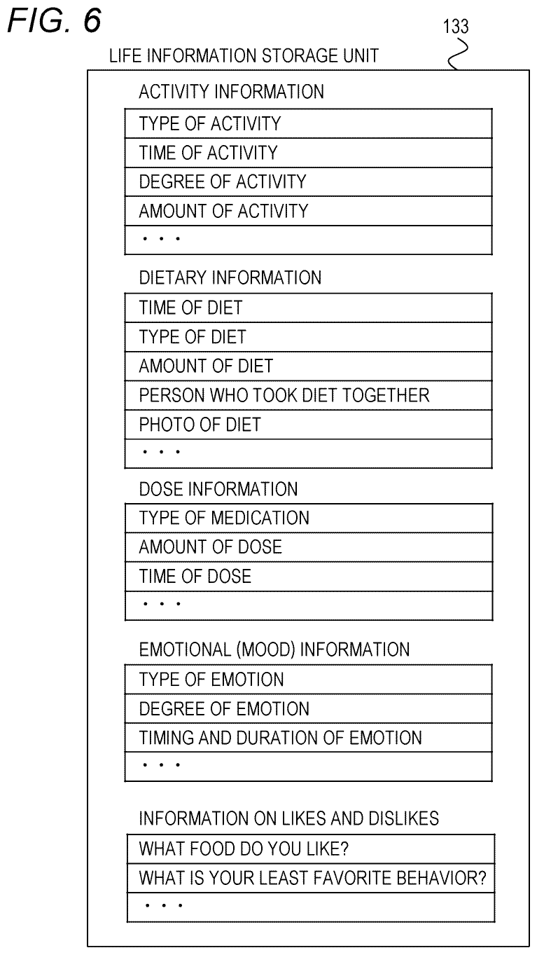

The life information storage unit 133 stores life information, an example of which is illustrated in FIG. 6, received by the life information reception unit 113. As illustrated in FIG. 6, the life information is information that occurs while a user lives for maintenance and improvement of health, or subjective information. As an example, the life information includes, but not limited to, activity information, dietary information, dose information, and emotional information. The activity information is configured from information such as the type of activity (including, but not limited to, exercise, sports, and other physical activities), the time of activity, the degree of activity, and the amount of activity, as an example. The dietary information is configured from information such as the time of diet, the type of diet, the amount of diet, and the person who took the diet together, as an example. The dose information is configured from information such as the type of medication, the amount of doses, and the time of doses, as an example.

The emotional information is configured from information such as the type of emotion (e.g., pleasant, unpleasant, joy, anger, sadness, pleasure, positive, neutral, or negative), the degree of emotion, and the timing and duration of emotion, as an example.

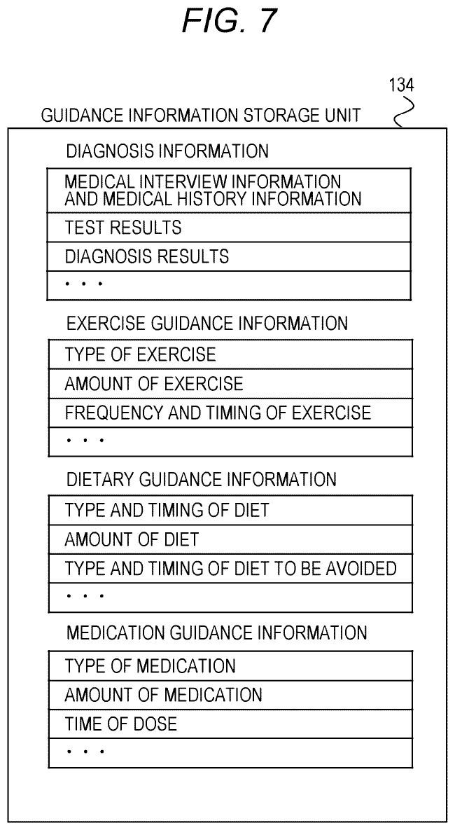

The guidance information storage unit 134 stores guidance information, an example of which is illustrated in FIG. 7, received by the guidance information reception unit 114. As illustrated in FIG. 7, the guidance information is information on activity that a medical professional such as a physician performs aiming at maintenance and improvement of health of a user, or health guidance to the user. As an example, the guidance information broadly includes, but not limited to, diagnosis information, exercise guidance information, dietary guidance information, and medication guidance information. The exercise guidance information is configured from information such as the type of exercise, the amount of exercise, and the frequency of exercise, as an example. The dietary guidance information is configured from information such as the type of diet, the amount of diet, and the type of diet to be avoided, as an example. The medication guidance information is configured from information such as the type of medication, the amount of medication, and the time of doses, as an example.

The above description is the data configuration of the server apparatus 1. It is noted that each piece of past data may be retained by being linked to the time the data was input by a user or a medical professional, or the time acquired with the sensor device.

Here, in the present embodiment, functions of the following functional units will be illustrated: the user information reception unit 111, the vital information reception unit 112, the life information reception unit 113, the guidance information reception unit 114, the symptom determination unit 115, the analysis unit 116, the presentation unit 117, and the model generation unit 118.

The user information reception unit 111 receives information on a user from the user terminal 3 via the network 2. The communication in the transmission and reception may be either wired or wireless, and any type of communication protocol may be used as long as the communication protocol allows for execution of intercommunication. The user information may be input from the medical professional terminal 5 via the network 2, based on information collected by the medical professional through interviews or questionnaires with the user. Further, a business operator who conducts business using the server apparatus 1 may collect the user information through interviews or questionnaires with a person responsible in the organization, and may input the information into the server apparatus 1 directly or input the information from a terminal of the business operator via the network 2.

The vital information reception unit 112 receives information on live data of the user from the sensor device 4 or the medical professional terminal 5 via the network 2. The communication in the transmission and reception may be either wired or wireless, and any type of communication protocol may be used as long as the communication protocol allows for execution of intercommunication.

The life information reception unit 113 receives information on various activities that the user performed in daily life from the user terminal 3 via the network 2. The communication in the transmission and reception may be either wired or wireless, and any type of communication protocol may be used as long as the communication protocol allows for execution of intercommunication.

The life information reception unit 113 may present a form to the user terminal 3 for inputting information on various activities performed in daily life. The life information reception unit 113 receives information input in the form by the user and stores the information in the life information storage unit 133. The activity includes, but not limited to, exercise, diet, medication, and sleep. The information on exercise may include the type of exercise, an indicator of the amount of exercise such as time and frequency, an indicator of the intensity of exercise, and the time of the day when exercise was performed. The information on diet may include the type of diet (e.g., menus, or ingredients), the amount of diet, the way diet is eaten (e.g., eating sooner, slower, alone, or with others), and the time of the day when diet is eaten. The information on medication may include the type of medication taken, and the time the medication was taken.

In addition, the life information reception unit 113 may present the form to the user terminal 3 at a predetermined time. The life information reception unit 113 presents a form for inputting, for example, the time around when diet generally ends (e.g., around 7:00 a.m.-8:00 a.m. for breakfast, or around 1:00 p.m. for lunch), what and how much diet was taken at a time predetermined by the user, and the type of medication and whether the user took medication. The form may allow selection of food categories such as meat, fish, vegetables, and fruit, with respect to the contents of diet. The form may allow selection of ranges such as 100 g or less, 100 g to 200 g, pieces or less, and 6 pieces to 10 pieces, with respect to the amount, and may allow selection from categories such as small amount, normal amount, and large amount. The life information reception unit 113 may present the form to the user based on information received from the sensor device 4. For example, in a case where heart rate that the vital information reception unit 112 receives from the sensor device 4 fluctuates beyond a certain degree, the life information reception unit 113 may present a form for inputting the type of exercise, the intensity of exercise, and the duration of exercise that have been performed, and the like. The form may be presented to the user together with a predicted value based on information received from the sensor device 4.

For example, when the vital information reception unit 112 receives information on such as GPS and a pedometer from the sensor device 4, the life information reception unit 113 may predict the type of exercise, the intensity of exercise, and the duration of exercise that have been performed based on the information, input numerical values in the form as a reference time and present the value to the user terminal 3, and receive transmission from the user. It is noted that the life information reception unit 113 may store the information of the prediction in the life information storage unit 133, and may further present the information of the prediction to the user, as activity performed, in a form that can be modified by the user.

Further, the life information reception unit 113 may present a form for inputting emotion to the user terminal 3. The emotion refers to the movement of feelings such as anger, fear, joy, and sadness. In order to remember what emotions were caused by various activities the user performed in daily life, the life information reception unit 113 may present the user terminal 3 with a form for inputting emotions at that time, in addition to information on the various activities performed in daily life. The form for inputting emotion may allow the user to select joy, anger, sadness, and pleasure as emotion, and may receive the degree of the emotion by input of a number or selection of a stage after the selection.

Further, the form may also allow the user to select icons for faces (e.g., a smiling face, or a crying face) and behavior (e.g., thumbs up, or thumbs down) representing feelings, or allow the user to input text about mood at that time and analyze whether the appearing words are positive or negative to estimate emotion, which are not limited to these. The life information reception unit 113 may also present only the form for inputting emotion to the user terminal 3, without linking to the activity.

In addition, the life information reception unit 113 may present the form for inputting emotion to the user terminal 3 when, as described later, the analysis unit 116 identifies performed behavior. This makes it possible to link the performed behavior to what feelings the user had as a result of the performed behavior.

It may be determined whether the user had positive or neutral (pleasant or not unpleasant) feelings or negative (unpleasant) feelings as a result of the performed behavior.

In addition, the life information reception unit 113 may present a form for inputting likes and dislikes to the user terminal 3. For example, the life information reception unit 113 presents the user with a form for inputting preferences for exercise, diet, sleep, and the like. Specifically, as an example, the life information reception unit 113 presents a question, which exercise would you like to do?, options such as walking, running, swimming, cycling, and others and free input fields, and a form that allows the user to input a degree of likes and dislikes with respect to each option by selection or inputting a number, such as a 5-point scale. In addition, as an example, the life information reception unit 113 presents a question, which of the following would you most like to avoid in your diet?, options such as eating less per diet, eating fewer diet per day, eating less salt, drinking less, and others and free input fields, and a form that allows the user to input a degree of likes and dislikes with respect to each option by selection or inputting a number, such as a 5-point scale. The contents of questions and a method for inputting a degree of likes and dislikes are not limited to these described above. The information on such likes and dislikes may be presented to a physician by the presentation unit 117, and those with a particularly high degree of liking may be presented as candidate actionable behavior, which is likely to be actionable for the user to work on with high motivation in guidance provided by the physician.

The guidance information reception unit 114 receives information on guidance that leads to the user's treatment and symptomatic alleviation, maintenance and improvement of health from the medical professional terminal 5 via the network 2. The communication in the transmission and reception may be either wired or wireless, and any type of communication protocol may be used as long as the communication protocol allows for execution of intercommunication.

The symptom determination unit 115 determines that symptoms of the user have improved or worsened based on the vital information. The symptom determination unit 115 uses, as an example, systolic blood pressure values and diastolic blood pressure values (included in information acquired with a biometric sensor) when the disease to be improved is hypertension, and uses LDL cholesterol values and HDL cholesterol values (included in information acquired with a biosensor) when the disease to be improved is dyslipidemia for determining a symptom. As an example, the symptom determination unit 115 determines as improvement of a symptom when the value of the item has fallen within a range of values judged to be inappropriate to a range of values judged to be appropriate, and determines as worsening of a symptom when the value of the item has fallen within the range of values judged to be appropriate to the range of values judged to be inappropriate, and stores time information at that point (when the symptom changes). The symptom determination unit 115 may determine that a sudden change in the symptom occurs when the value of the item has fluctuated rapidly. It is noted that the values used by the symptom determination unit 115 to determine symptoms are not limited to the above-described values, and appropriate indicators should be set depending on other diseases or risks to be analyzed. Further, the symptom determination unit 115 may determine that a change in a value that is determined to be medically inappropriate, such as a high or low value even within an appropriate range, is worsening of the symptom.

The analysis unit 116 analyzes actionable activities for a user. The analysis unit 116 identifies, for example, behavior that the user can continuously perform as actionable behavior based on the vital information or the life information.

The analysis unit 116 identifies behavior repeated in ranges of minutes, hours, days, weeks, months, or the like based on the vital information or the life information. For example, the analysis unit 116 acquires average pulse rate of the user in a calm state from pulse rate information included in the vital information. Next, the analysis unit 116 identifies that the user has exercised (performed behavior) when the pulse rate exceeding the pulse rate in a calm state continues intermittently for several minutes to several hours. Further, the analysis unit 116 estimates the type of exercise (e.g., walking, running, swimming, or weight training), the average intensity and duration of the exercise based on information on how much the pulse rate exceeds the pulse rate in a calm state, how intermittently the pulse rate continues, and other information such as GPS information on movement of the location of the user. It is noted that when the exercise is linked to the life information, the analysis unit 116 may change information of the life information to the contents that has been estimated. Further, for example, the analysis unit 116 identifies that the user is in a sleep state when the user's motion information (stored in the vital information storage unit 132), which is acquired by a multi-axis acceleration sensor or the like included in the sensor device 4, is hardly observed for a certain period of time (e.g., 10 minutes, or 30 minutes). Further, the analysis unit 116 may determine that in the sleep state, the user is in a deep sleep during a period of time when the amount of activity of the user is significantly low and that the sleep is shallow when the amount of activity is significantly increases for a certain period of time, and identify the activity as performed behavior.

The analysis unit 116 acquires the type of diet from the dietary information included in the life information and identifies the type of diet as performed behavior. The analysis unit 116 determines ingesting or not ingesting a specific ingredient in more or less than a specific amount as performed behavior based on information acquired from a database (which may be provided in the server apparatus 1, or data may be acquired from the Internet or the like) that has information on such as ingredients and amounts included in the diet of the type. Regarding the type and the amount of diet, the analysis unit 116 may analyze photos of diet included in the dietary information and estimate the menu and the amount of diet. Similarly, the analysis unit 116 may identify which type of medication is being taken, to what degree, and at what time, as performed behavior based on the dose information included in the life information.

Further, when the performed behavior has been repeated more than a specified number of times during a certain period of time, such as three or more times a day, three or more times a week, or three or more times a month, or when the performed behavior has been repeated for a specified number of days, such as three consecutive days, one consecutive week, or one consecutive month, the analysis unit 116 determines that the exercise is actionable behavior for the user.

Further, the analysis unit 116 may determine that the performed behavior is actionable behavior based on information on emotion input by the user in the form for inputting emotion at that time, which is presented to the user by the life information reception unit 113 after the performed behavior, and set a higher priority than other actionable behavior. For example, when the number of times the user inputs non-negative emotion after walking exceeds a certain number, such as five or more times, the analysis unit 116 determines that walking is actionable behavior for the user. In addition, for example, when the number of times the user inputs negative emotion after a diet, which has been identified as performed behavior, with fish as the main exceeds a certain number of times, the analysis unit 116 may determine that a diet with fish as the main is not actionable behavior for the user.

Further, the analysis unit 116 identifies activities that the user can continuously stop as actionable behavior based on the vital information or the life information.

For example, as described above, based on the vital information or the life information, the analysis unit 116 identifies performed behavior such as exercise and diet that is performed continuously or intermittently, but that has been no longer performed or that has been performed less frequently at a certain time, as actionable behavior.

In addition, for example, as described above, after the performed behavior such as exercise and diet that is performed continuously or intermittently was performed, when the emotion that has been input in a form presented by the life information reception unit 113 is such as non-negative emotion or emotion that was previously negative emotion but now changed to non-negative emotion, the analysis unit 116 may identify the performed behavior as actionable behavior based on the vital information or the life information, and set a higher priority than other actionable behavior.

Further, the analysis unit 116 identifies behavior that can be performed instead, if the user stops the performed behavior, as actionable behavior, based on the vital information or the life information.

For example, as described above, the analysis unit 116 may identify the performed behavior such as exercise and diet that the emotion input in the form presented by the life information reception unit 113 has been positive or non-negative and the like after the behavior is performed in an unexpected manner, as actionable behavior based on the vital information or the life information, and set a higher priority than other actionable behavior.

In addition to the user inputting information on emotion as subjective information in the form presented by the life information reception unit 113, the analysis unit 116 may also read information acquired through the sensor device 4, such as the amount of perspiration, the number of beats, and the amount of stress hormones, from the vital information storage unit 132, estimate emotion such as positive, neutral, and negative, link the emotion to the performed behavior, and determine whether the performed behavior is actionable.

Further, when the symptom determination unit 115 determines that a symptom of the user has improved or worsened based on the vital information during or after the time the performed behavior identified by the analysis unit 116 was performed, the analysis unit 116 may identify the performed behavior as symptom-related actionable behavior that may be related to the change in the symptom, and set a higher priority than other actionable behavior. The time may be a specified time (e.g., 10 minutes, 30 minutes, 60 minutes, 4 hours, 12 hours, or 24 hours, which can be set individually depending on the target disease or symptom). After the performed behavior was performed, and the more times the symptom determination unit 115 determines that the symptom of the user has improved or worsened, the higher the correlation, and the performed behavior is identified as symptom-related actionable behavior. It is noted that the analysis unit 116 may lower the priority of symptom-related actionable behavior that is highly correlated with worsening symptoms.

Further, the analysis unit 116 may determine whether the user has followed the guidance of a physician by comparing the treatment information with the performed behavior identified by the analysis unit 116. Specifically, when the treatment information includes, for example, walking for 60 minutes or more at least three times a week, and when walking for 60 minutes or more has been performed at least three times a week in the performed behavior identified by the analysis unit 116, the user is determined to have followed the guidance. When the user has been walking for 60 minutes or more only once a week, the user is determined to have failed to follow the guidance.

Further, the analysis unit 116 may statistically estimate actionable behavior. The type of analysis used by the analysis unit 116 may include classification, regression, correlation analysis, calculation of feature amount importance, and clustering. These statistical models can be implemented using implementations generally used in statistics, which will not be described in detail herein.

The analysis unit 116 analyzes whether the actionable behavior described above is correlated with improvement of a symptom. The analysis unit 116, for example, statistically analyzes life information performed up to the time of symptom change by going back a specified time (e.g., 10 minutes, 30 minutes, 60 minutes, 4 hours, 12 hours, or 24 hours) from the time of the symptom changed, and estimates symptom-related actionable behavior. In the case of learning, input data used for learning is at least the vital information and the life information, and teacher data is life information prior to the time when the symptom determination unit 115 determined symptom fluctuation in the life information.

The presentation unit 117 presents the performed behavior, the actionable behavior, the symptom-related actionable behavior, and the candidate actionable behavior to the user terminal 3 or the medical professional terminal 5. The presentation unit 117 presents the performed behavior, the actionable behavior, and the symptom-related actionable behavior along a time axis. The presentation unit 117 may also present to the medical professional a check box to check the symptom-related actionable behavior and the candidate actionable behavior that the user was actually guided to perform, and the checked behavior is stored in the server apparatus 1 as information with a correct answer label given by the medical professional. The presentation unit 117 may also present actionable behavior (accompanied by non-negative emotion) and symptom-related actionable behavior for which the analysis unit 116 has set a higher priority to the medical professional in a conspicuous and distinguishable manner, such as by display at the top of a screen to be presented, display with a higher priority, changing the color, and changing the font size. However, methods for presentation are not limited to these. Further, the presentation unit 117 does not have to present symptom-related actionable behavior for which the analysis unit 116 has set a lower priority. It is noted that variation in indicators of a target symptom included in the vital information may be presented together with the behavior. This will make it easier for medical professionals to consider guidance policies for users. Further, activities output by a prediction model generated by a model generator 118 described below may be presented as actionable behavior or symptom-related actionable behavior.

The presentation unit 117 may first present a screen to be presented to the user terminal 3 to the medical professional terminal 5, receive edits such as corrections and additions from the medical professional, and present information reflecting the edits to the user terminal 3.

The presentation unit 117 may also present a form to the user terminal 3 to input whether the user himself/herself is actionable from among the performed behavior, the actionable behavior, the symptom-related actionable behavior, and the candidate actionable behavior that have been presented. In this case, the presentation unit 117 can present the user with a form that receives the order of ease of performing and desirability for performing each of the performed behavior, the actionable behavior, the symptom-related actionable behavior, and the candidate actionable behavior by numerical selection or input, selection of an icon indicating intent such as thumbs up and thumbs down, or the like.

The model generation unit 118 may generate a prediction model for predicting actionable behavior for a user group such as constitution of the user and characteristics of performed behavior by statistical methods such as learning, based on the performed behavior, the actionable behavior, the symptom-related actionable behavior, and the candidate actionable behavior of multiple users, as well as user information and the guidance information. Methods for generating the prediction model used by the model generation unit 118 may include classification, regression, correlation analysis, calculation of feature amount importance, and clustering. These statistical models can be implemented using implementations generally used in statistics, which will not be described in detail herein. Input data to the model from which relationships can be derived, generated by these methods, is the performed behavior, the actionable behavior, the symptom-related actionable behavior, the candidate actionable behavior, the user information, and the guidance information. The medical professional may assign a correct answer label as a teacher label to the symptom-related actionable behavior or the candidate actionable behavior. The teacher label may also be the behavior that the user has rated as preferred, as received by the presentation unit 117 for the symptom-related actionable behavior or the candidate actionable behavior.

Further, as described above, the model generation unit 118 may generate the prediction model by statistical methods such as learning, a specific machine learning model will be described as an example. The prediction model generated by the model generation unit 118 may be a model that predicts performed behavior that is continuously performed. In this case, input data to the machine learning model is the performed behavior and user information, and output data is the performed behavior that is continuously performed, but are not limited to these. The prediction model generated by the model generation unit 118 may also be a model that predicts behavior accompanied by positive emotion or behavior accompanied by non-negative emotion in the performed behavior. In this case, input data to the machine learning model is the performed behavior, the emotional information, and the user information, output data is the performed behavior accompanied by positive emotion or the performed behavior accompanied by non-negative emotion, but are not limited to these. The prediction model generated by the model generation unit 118 may also be a model that predicts the symptom-related actionable behavior. In this case, input data to the machine learning model is the performed behavior, the vital information, and the user information, output data is the symptom-related actionable behavior, but are not limited to these. It is noted that in addition to the user information, the type, the amount, and the degree of specific performed behavior (e.g., exercise) may be used as a feature amount, or the type and the amount of diet and doses and even symptoms and disease names may be used.

Further, the model generation unit 118 may generate the prediction model based on, for example, knowledge of the medical industry. In this case, the model generation unit 118 may use the behavior that is first generally presented to patients in the medical industry for patients with a particular disease or medical condition, as the output of the prediction model. The model generation unit 118 may also present a form for scoring and prioritizing the behavior that is first generally presented to patients in the medical industry in terms of effect, effectiveness, and ease of performing for the user to the medical professional terminal 5, and use the behavior that many medical professionals give a high score or a high priority, as the output of the prediction model. The model generation unit 118 may also present to the medical professional terminal 5 a form for inputting behavior suggested by the medical professional for patients with a specific disease or medical condition and, for example, use the behavior that a large number of the same responses has been made, as the output of the prediction model, based on information on responses collected through input to the form. Further, the server apparatus 1 may receive literature information such as medical and health articles and reviews, and the model generation unit 118 may generate the prediction model based on the literature information. For example, the model generation unit 118 may use the behavior that appears in more than a certain number of literature and is recommended for patients with a specific disease or medical condition, the behavior citations of which in the literature in which the behavior is described exceed a certain number of times, the behavior that an impact factor of a journal in which the literature is published exceeds a certain value, or the like as the output of the prediction model.

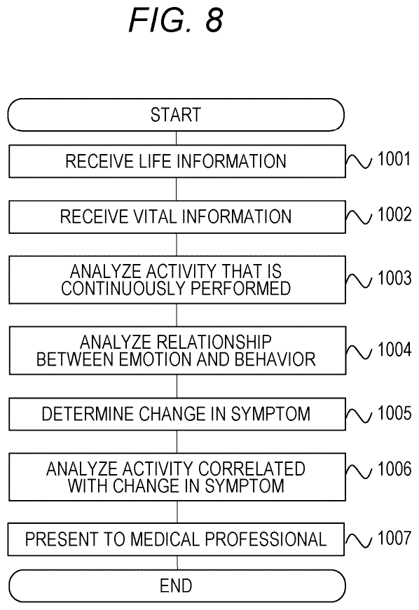

A typical flow of processing of the present embodiment will be described by using FIG. 8. First, the life Information reception unit 113 receives life information (1001). Next, the vital information reception unit 112 receives vital information (1002). The order of reception of the life information (1001) and reception of the vital information (1002) may be interchanged. The analysis unit 116 analyzes activities that are continuously performed and identifies actionable behavior based on the life information (1003). Further, the analysis unit 116 analyzes information of emotion linked to the activities together and prioritizes the actionable behavior (1004). The symptom determination unit 115 determines changes in symptoms based on the vital information (1005). The analysis unit 116 analyzes the activities that were being performed prior to the time when the symptom change was determined and identifies symptom-related actionable behavior (1006). The presentation unit 117 presents the actionable behavior and the symptom-related actionable behavior (1007).

Preferred embodiments of the present disclosure have been described with reference to the accompanying drawings, but the technical scope of the present disclosure is not limited to such examples.

It is apparent to those skilled in the technical field of the present disclosure that various examples of changes or modifications can be conceived of within the scope of the technical idea described in the appended claims, and these also naturally fall within the technical scope of the present disclosure.

The devices described herein may be implemented as stand-alone devices or as a plurality of devices some or the entirety of which is connected by a network (for example, a cloud server). For example, the CPU 101 and the storage device 103 of the server apparatus 1 may be implemented by different servers connected via a network to each other.

The series of processes carried out by the apparatus described in the present specification may be implemented using any of software, hardware, and a combination of software and hardware. A computer program for implementing the functions of the server apparatus 1 according to the present embodiment can be fabricated and implemented in a computer or the like.

In addition, a computer-readable recording medium that stores such a computer program can also be provided.

The recording medium is, for example, a magnetic disk, an optical disc, a magneto-optical disk, or a flash memory. The computer program described above may be distributed, for example, via a network without using a recording medium.

The processes described herein using a flowchart do not necessarily have to be executed in the order illustrated. Some of the processing steps may be performed in parallel. Additional processing steps may be employed, and some processing steps may be omitted.

The advantageous effects described herein are illustrative or exemplary only and not limiting. In other words, the technology of the present disclosure may produce other effects that are obvious to those skilled in the art from the description herein, either together with or in place of the above-described advantageous effects.

### REFERENCE SIGNS LIST

1 Server apparatus
2 Network
3 User terminal
4 Sensor device
5 Medical professional terminal
101 CPU
102 Memory
103 Storage device
104 Communication interface
105 Input device
106 Output device
111 User information reception unit
112 Vital Information reception unit
113 Life Information reception unit
114 Guidance information reception unit
115 Symptom determination unit
116 Analysis unit
117 Screen presentation unit
118 Model generation unit
119 Diagnosis support unit
120 Message transmission unit
131 User information storage unit
132 Vital information storage unit
133 Life information storage unit
134 Guidance information storage unit

1. A health support apparatus for supporting symptomatic alleviation or maintenance and improvement of a physical condition of a user, the health support apparatus comprising:
a life information reception unit configured to receive life information including information on activity that the user performs in daily life;
an analysis unit configured to identify actionable behavior that the user is able to perform in the activity; and
a presentation unit configured to present the actionable behavior to the user or a medical professional.

2. The health support apparatus according to claim 1,
wherein
the life information includes information on emotion of the user when the activity has been performed,
the analysis unit increases priority of actionable behavior accompanied by non-negative emotion in the actionable behavior, and
the presentation unit presents the actionable behavior for which the analysis unit has increased priority in a manner distinguishable from the other actionable behavior.

3. The health support apparatus according to claim 1, further comprising:
a vital information reception unit configured to acquire vital information of the user; and
a symptom determination unit configured to determine a change in a symptom of the user from the vital information, wherein
the analysis unit identifies the actionable behavior performed before a time when the symptom determination unit determines that the symptom has improved, as symptom-related actionable behavior, and
the presentation unit presents the symptom-related actionable behavior in a manner distinguishable from the actionable behavior.

4. The health support apparatus according to claim 1, wherein the analysis unit statistically estimates the actionable behavior.

5. The health support apparatus according to claim 1, further comprising:
a user information reception unit configured to receive user information on an attribute of the user; and
a model generation unit configured to estimate the actionable behavior of the user.

6. The health support apparatus according to claim 5, wherein
the model generation unit uses the actionable behavior as correct data and generates a prediction model in which input data is the user information and the life information and output is behavior that the user is able to perform, and
the presentation unit presents the behavior output by the prediction model.

7. The health support apparatus according to claim 5, wherein
the model generation unit uses the symptom-related actionable behavior as correct data and generates a prediction model in which input data is the user information and the life information and output is behavior that the user is able to perform, and
the presentation unit presents the behavior output by the prediction model.

8. A health support system for supporting symptomatic alleviation or maintenance and improvement of a physical condition of a user, the health support system comprising:
a life information reception function to receive life information including information on activity that the user performs in daily life;
an analysis function to identify actionable behavior that the user is able to perform in the activity; and
a presentation function to present the actionable behavior to the user or a medical professional.

9. A health support method for supporting symptomatic alleviation or maintenance and improvement of a physical condition of a user,
the health support method performed by a processor comprising:
a life information receiving step of receiving life information including information on activity that the user performs in daily life;
an analyzing step of identifying actionable behavior that the user is able to perform in the activity; and
a presenting step of presenting the actionable behavior to the user or a medical professional.
